(19) United States
(12) Patent Application Publication
(10) Pub. No.: US 2024/0156436 A1
(43) Pub. Date: May 16, 2024

(54) Ultrasound Blanket Device

(71) Applicant: Natasha Elizabeth Kelly, Ansonia, CT (US)

(72) Inventor: Natasha Elizabeth Kelly, Ansonia, CT (US)

(21) Appl. No.: 17/929,385

(22) Filed: Nov. 15, 2022

(51) Int. Cl. A61B 8/00; A47G 9/02

(52) U.S. Cl. CPC A61B 8/4494; A47G 9/0223; A61B 8/56; A47G 2009/001

(57) ABSTRACT

The invention is a computerized ultrasound blanket scanning device using sonographic waves allowing contemporaneous monitoring of fetal-maternal vital signs, and pertinent laboratory following the development of the fetal/maternal physiology in the gravid phase. Innovatively, the surface area assessed, is determined by the computerized ultrasound blanket instead of a manual transducer probe, leading to user dependent errors/misinformation. The challenges include maintaining the sonographic frequencies low enough to prevent cavitation of biochemical molecules within the fetus and Mother via heating, and preventing short circuit of pico-nano transducers keys over time. The solution entails replacing the transducers every 6-12 months using current upgrades of technologically advanced transducers, with progressively smaller transducers over time.

### FIELD

The present application relates to a technological innovation in the field of ultrasonographic sound waves. More particularly, the present technology pertains to a computerized ultrasound blanket scanning device which uses sonographic waves for fetal maternal monitoring.

### BACKGROUND

The United States of America ranks number 1 for developed countries regarding fetal maternal mortality rate for 2022. Additionally, black, brown, and indigenous women fetal maternal mortality rate is 3-5 times higher than white women. To address this discrepancy in the healthcare of maternal women, and minimize the fetal maternal mortality rate in marginalized and socioeconomic disadvantaged communities, the computerized ultrasound blanket device will utilize Telemedicine monitoring with the aide of Doulas for the dissemination, and delivery of culturally relevant and appropriate fetal maternal care.

### SUMMARY

In one embodiment the technological innovation is a computerized ultrasound blanket scanning device which uses sonographic waves that allows for timely and continuous monitoring of fetal-maternal vital signs and pertinent laboratory to follow growth and development of fetus and maternal changing physiology during this period of time. The computerized QWERTY keyboard like device with nano-pico transducer keys will cover the gravid abdomen anatomically from subcostal region below the 12^{th} ribs to symphysis pubis anteriorly, and posterior axillary line laterally.

This level of close and skilled monitoring of physiology will eliminate operator errors via transducer manipulation, since the blanket ultrasound device scans/covers surface area of interest. Thus, with the aid of a culturally relevant and appropriate Doula, there will be minimization the current fetal-maternal mortality rate in black, brown and indigenous populations. Additionally, extensive training with the device is eliminated since one does not use a transducer to move over the surface area of interest, and the computerized QWERTY keyboard like keys are used to generate pertinent B-mode sonographic images and vital signs.

The ultrasound blanket scanning device allows for Telemedicine monitoring of the fetus and Mother during this critical period of great physiological change. The data harnessed from this level of acuity care will greatly improve the care of fetus and Mother with extrapolation of data nationally.

Harnessing of real time data mining with improved affordability, accessibility of fetal-maternal care. Additionally, further objects, features, advantages, and properties of the device according to the present application will become apparent from the detailed description.

### BRIEF DESCRIPTION OF DRAWING

In the following detailed portion of the present description, the teachings of the present application will be explained in more detail with reference to the example embodiment shown in the drawing, in which:

FIG. 1 is an illustration of a network diagram of an example computer system or computing device that can be utilized in various scenarios, according to the embodiment of the present technology with the depiction of 9 abdominopelvic regions with a total of 216 sequential nano-pico transducer wafer keys/probes.

### DETAILED DESCRIPTION

In the following detailed description, the device according to the teachings for this application in the form of a computerized ultrasound blanket device for fetal maternal monitoring will be described by the embodiment.

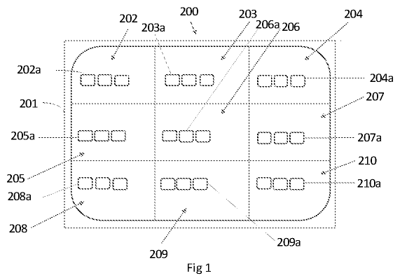

The embodiment of the computerized ultrasound blanket device 200 is illustrated in FIG. 1. The thin computerized QWERTY keyboard like device 200 comprises a housing 201 with a total of 216 nano-pico transducer keys which will cover the gravid abdomen anatomically from subcostal region below the 12^{th} ribs to symphysis pubis anteriorly, and posterior axillary line laterally and be 5 mm to 1 cm thick, 24 inches wide and 19 inches long. The thin computerized QWERTY keyboard like device 200 will cover the surface area of the gravid abdomen, which correlates to the 9 abdominopelvic intra-abdominal anatomic regions housing 202, 203, 204, 205, 206, 207, 208, 209 and 210, with 3 column sections in each horizontal subsection row, 202/203/204, then 205/206/207 then 208/209/210. For example, horizontal row subsection 1 comprises right hypochondriac region housing 202, epigastric region housing 203, and left hypochondriac region housing 204. Horizontal row subsection 1 will thus comprise 3 columns each with 24 nano-pico transducer keys, starting with Q1 key, housing 202a (Q1-Q24), 203a (Q25-Q48) and 204a (Q49-Q72). Thus, each horizontal subsection row will have a total of 72 nano-pico transducer keys. Horizontal subsection row 2 will house 205a (Q73-Q96), 206a (Q97-Q120), 207a (Q121-Q144), and horizontal subsection row 3 will house 208a (Q145-Q168), 209a (Q169-Q192), 210a (Q193-Q216) see FIG. 1. Consequently, the gravid abdomen will require a minimum of 216 QWERTY like nano-pico transducer keys for the computerized digital keyboard.

The teaching of the present application has numerous advantages. It should be noted that this is not an exhaustive list, and there may be other advantages which are not described herein. What is innovative/advantageous, is the surface area to be assessed with the ultrasound blanket scanning device instead of running a transducer along and over the abdomen several times, which can lead to a lot of user dependent errors and misinformation. It is yet another advantage that, pH, blood pressure, and oxygen saturation vital signs will be calculated from algorithmic Doppler flow formula from bimodal ultrasonic imaging using both continuous and pulsed sonographic waves. The challenges will be to keep the sonographic frequencies low enough to prevent cavitation of biochemical molecules within the fetus and Mother via heating and preventing short circuit of pico-nano transducer over time. The solution would be to replace the transducers every 6-12 months via current upgrade of technological more advanced transducers, and using progressively smaller transducers over time. This level of close and skilled monitoring of physiology will eliminate operator errors via transducer manipulation, since the blanket ultrasound device scans/assesses surface area of interest.

Features described in the proceeding description may be used in combinations other than the combinations explicitly described.

Whilst endeavoring in the foregoing specification to draw attention to those features of the invention believed to be of particular importance, it should be understood that the Applicant claims protection in any patentable feature or combinations of features herein before referred to and/or shown in the drawings whether or not particular emphasis has been placed thereon.

The term “comprising” does not as used in the claims does not exclude other elements or steps. The terms “a” or “an” as used in the claims does not exclude a plurality. A unit or other means may fulfill the functions of several units or means recited in the claims

1. Computerized ultrasound blanket device comprising of hypoallergenic microfiber rubber/Teflon material.

2. The computerized ultrasound blanket device of claim 1 is comprised of a thin blanket of hypoallergenic microfiber rubber/Teflon material that sandwiches nao-pico transducer keys/probes.

3. The computerized ultrasound blanket device in claim 2 comprises of 216 nano-pico transducer QWERTY keyboard like keys/probes.

4. The computerized ultrasound device in claim 3 comprises 3 anatomic abdominopelvic sections.

5. The computerized ultrasound device of claim 4 comprises 72 nano-pico transducer keys/probes in each anatomic subsection totaling 216 keys.

6. The computerized ultrasound device of claim 5 comprises of nano-pico transducer keys/probes labeled Q1 to Q216 in a QWERTY like keyboard fashion.

7. The computerized ultrasound device of claim 6 will be activated/turned on by a USB port or Bluetooth

8. The computerized ultrasound device in claim 7 will transmit data via an USB port or Blue tooth to Women's Health Center for tertiary level clinical assessment of marginalized maternal patients in underserved areas and populations.

9. The computerized ultrasound device in claim 8 will collect and collate data using the nano-pico transducer QWERTY keyboard like keys/probes

10. The computerized ultrasound device of claim 9 can algorithmically shuffle the nano-pico transducer QWERTY like keyboard keys/probes to be used for sonochemistry and give an improved Doppler B-mode sonographic frequency.
